(19) United States
(12) Patent Application Publication
(10) Pub. No.: US 2008/0293946 A1
(43) Pub. Date: Nov. 27, 2008

(54) Process for Preparing Zolpidem Hemitartrate and Tartrate Polymorphs

(76) Inventor: Brian K. Cheng, Chesterfield, MO (US)

Correspondence Address: Mallinckrodt Inc., 675 McDonnell Boulevard, HAZELWOOD, MO, 63042, US

(21) Appl. No.: 12/067,816

(22) PCT Filed: Sep. 19, 2006
(86) PCT No.: PCT/US06/36648

§ 371 Date: Mar. 24, 2008

Related U.S. Application Data
(60) Provisional application No. 60/723,076, filed on Oct. 3, 2005.

(51) Int. Cl. C07D 471/04

(52) U.S. Cl. 546/121

(57) ABSTRACT

A method for preparing a polymorph of a hemitartrate salt of a compound having the structure: comprising dissolving a free base form of the compound in a liquid medium comprising an alcohol and a tartrate derivative to form a solution comprising the compound, the alcohol, and the tartrate derivative; heating the solution to a temperature sufficient to dissolve the compound and the tartrate derivative; and cooling the solution to a temperature sufficient to precipitate the hemitartrate salt of the compound.

### FIELD OF INVENTION

The present invention relates to the preparation of a variety of polymorphs of a pharmaceutical compound, and more specifically, the invention relates to a process for making selected zolpidem hemitartrate and tartrate polymorphs.

### BACKGROUND OF THE INVENTION

The benzodiazepine family of hypnotics and sleep aids includes triazolam (Halcion®), alprazolam (Xanax®), and diazepam (Valium®), among many others. The members of the benzodiazepine family are known to have anxiolytic, sedative, hypnotic, anticonvulsant, and muscle relaxant properties. Zolpidem hemitartrate, which is marketed as Ambien®, Stilnox®, and Stilnoct®, is a non-benzodiazepine drug which is part of the imidazopyridine family, but Zolpidem hemitartrate has been found to have similar pharmacological effects as the benzodiazepines.

Zolpidem hemitartrate is known to exist in several polymorphs, among which are known the A, B, C, D, E, F, G, and H forms. See WO 01/80857 A1 by Teva Pharmaceutical Industries, Ltd., the disclosure of which is hereby incorporated by reference in its entirety for all purposes. Polymorphism refers to the occurrence of different crystalline forms of a particular drug compound. Many pharmaceuticals exist in different solid crystalline forms or as amorphous solids. A particular solid mass of a pharmaceutical may include a mixture of one or more crystalline polymorphs and/or amorphous forms. The particular polymorph of a pharmaceutical depends upon process conditions, such as processing from an aqueous solution, an organic solution, or mixtures of solvents. Other factors affecting the polymorph obtained include temperature, pressure, and atmospheric composition. It has been found that exposure to organic volatiles may cause a transition from one polymorph to another.

Polymorphic stability appears to depend upon environmental conditions and/or selected solvent systems. By this, it is meant that a particular crystalline form of a compound may precipitate under one set of conditions, i.e., solvent system, temperature, and atmosphere, while a different crystalline form may precipitate under a different solvent system, temperature, and atmosphere. Changing solvents, temperature, and/or atmosphere may cause a transition from one polymorph to another.

Control of pharmaceutical polymorphism is important in the industry because physical properties such as particle size, shape, flow characteristics, melting point, degree of hydration or solvation, and caking tendency affect such factors as chemical processing, material handling, compatibility with excipients, segregation in the blend, dissolution rate of a drug in aqueous media, and stability of the final dosage form. Changes in chemical properties due to polymorph transitioning can affect drug degradation induced by environmental factors such as heat, light, moisture, mechanical handling, oxygen, and interaction with excipients. Thus, the overall adverse effects of polymorph transitioning include production inefficiencies (time and cost), reduced product quality, and instability of the drug in tablet/pill form.

Therefore, there exists a need for methods which can selectively yield desired polymorphs for a given drug formulation. The benefits include simplification of the process and manufacturing cost savings for both the pharmaceutical active ingredient and the finished dosage form.

Teva Pharmaceutical Industries, Ltd., WO 01/80857 A1, has disclosed a method for converting zolpidem polymorphs by solvating with water, methanol, ethanol, propanol, butanol, ethylacetate, and the like. The results from the disclosed method often are irreproducible, particularly in production scale. In the disclosure, polymorph E was converted from other polymorphs isolated from water or solvent contact. The extra chemical processing steps and the need for solvent recovery steps required in the method can increase the production cost. Furthermore, some polymorphs are particularly difficult to process because of their physical properties.

### SUMMARY OF THE INVENTION

Among the various aspects of the present invention may be noted the provision of a process for preparing selected polymorphs of zolpidem hemitartrate. Particularly, a process is provided which allows for the preparation of desired polymorphs directly from the reaction between zolpidem free base and L-(+)-tartaric acid without the need for first preparing a particular polymorph and then processing that polymorph to the desired polymorph or isolating a desired polymorph from a mixture of polymorphs.

Briefly, therefore, the present invention is directed to a method for preparing a desired polymorph of a hemitartrate salt of a compound having the structure:

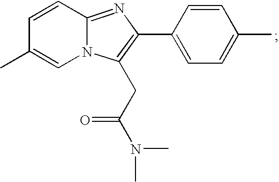

comprising the steps of dissolving a free base form of the compound in a liquid medium comprising an alcohol and a tartrate derivative to form a solution comprising the compound, the alcohol, and the tartrate derivative; heating the solution to a temperature sufficient to dissolve the compound and the tartrate derivative; and cooling the solution to a temperature sufficient to precipitate the hemitartrate salt of the compound.

Other objects and aspects of the invention will be, in part, pointed out and, in part, apparent hereinafter.

### DETAIL DESCRIPTION OF THE PREFERRED EMBODIMENT(S)

The present invention provides a novel process for the preparation of polymorphs of zolpidem hemitartrate. Zolpidem (N,N,6-Trimethyl-2-(4-methylphenyl)-imidazo[1,2-a]pyridine-3-acetamide) has the general structure (1):

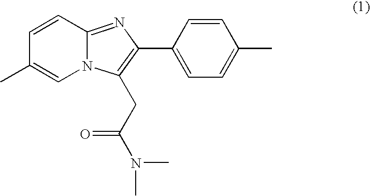

Zolpidem hemitartrate is typically prepared as a salt of L-(+)-tartaric acid ((2R,3R)2,3-dihydroxybutanedicarboxylic acid). The molar ratio of zolpidem to L-tartrate in the hemitartrate salt is approximately 2:1. Conventional syntheses yield a mixture of the known polymorphs of zolpidem hemitartrate. Accordingly, the isolation of a preferred polymorph requires extra chemical processing steps, which can add to the overall cost of producing a marketable product. According to the method of the present invention, selected polymorphs can be prepared directly from the free base, without the extra steps involved in isolating a particular polymorph from a mixture of zolpidem hemitartrates, thus yielding efficiencies in the production of formulations of zolpidem hemitartrate in terms of both lower cost and increased throughput.

The process of the present invention involves preparing a selected polymorph of zolpidem hemitartrate by dissolving zolpidem free base and an L-(+)-tartaric acid derivative in an alcohol solvent system, heating the solution, cooling the solution, and isolating the zolpidem hemitartrate polymorph.

Zolpidem free base can be dissolved in an alcohol solvent system. Suitable alcohols for the preparation of zolpidem hemitartrate polymorphs include methanol, ethanol, isopropanol, and n-propanol. Preferably, the zolpidem free base is substantially soluble in the alcohol chosen.

An L-(+)-tartaric acid derivative is added to form the selected zolpidem hemitartrate polymorph. The derivative can be dissolved directly in the alcohol solution comprising zolpidem free base. Alternatively, a separate alcohol solution can be prepared comprising the L-(+)-tartaric acid derivative. The L-(+)-tartaric acid derivative solution can then be added to the alcohol solution comprising the zolpidem free base. Preferred sources of L-(+)-tartrate include L-(+)-tartaric acid; monobasic salts of L-(+)-tartaric acid such as potassium L-(+)-tartrate monobasic salt, sodium L-(+)-tartrate monobasic salt, and ammonium L-(+)-tartrate monobasic salt; and dibasic salts of L-(+)-tartaric acid such as potassium L-(+)-tartrate dibasic salt, sodium L-(+)-tartrate dibasic salt, and ammonium L-(+)-tartrate dibasic salt. Preferably, the L-(+)-tartaric acid derivative is added such that a molar ratio of zolpidem free base to the L-(+)-tartaric acid derivative may be between about 2.5:1 and about 2:1, more preferably about 2:1.

Water can be added to the alcohol solution comprising the zolpidem free base and L-(+)-tartaric acid derivative. Preferably, the water is added such that a volume ratio of alcohol to water may be between about 10:1 and about 1:1, more preferably between about 5:1 and about 1:1, even more preferably about 2:1. The concentration of the zolpidem and tartaric acid solids is not narrowly critical to the efficacy of the invention. The solubility of tartaric acid in water is about 4%; therefore, preferably, the solution volume is sufficient to dissolve the solids. However, large solution volumes may decrease the yield; therefore, the solution volume is limited to achieve satisfactory yield. Accordingly, the concentration of zolpidem free base can preferably be between about 0.30 M and about 0.35 M.

The solution is heated to dissolve the solids and induce a reaction between the zolpidem free base and the L-(+)-tartaric acid derivative. The reaction results in the protonation of the zolpidem base and subsequent molecular coupling of two protonated zolpidem molecules per one L-(+)-tartrate molecule to form zolpidem hemitartrate. Heating is preferably to a temperature between about 25° C. and about 85° C., more preferably between about 50° C. and about 70° C. Heating may be accompanied by agitation, for example, stirring. Agitation can be accomplished by mechanical stirring. Heating may occur before, after, or concurrently with the addition of water. For example, the zolpidem free base and L-(+)-tartaric acid can be dissolved in a solution comprising an alcohol and water as a solvent, which is then heated. Alternatively, the zolpidem free base and L-(+)-tartaric acid can be dissolved in alcohol, which is heated before the addition of water.

The heating can be followed by rotary evaporation, distillation, azeotroping, or spray drying. For example, the solution can be distilled or azeotroped until an end point is reached, indicated by monitoring the temperature of the vapor. Depending upon the alcohol, the solvent combination of water and alcohol can form an azeotrope. For example, it is known that water:ethanol and water:propanol systems form azeotropes, while water:methanol systems do not. With respect to the methanol:water solvent system, methanol can be substantially removed by distillation. Substantial methanol removal can be indicated by a vapor temperature of about 94° C. Where the solvent system comprises ethanol:water or propanol:water, the system is preferably azeotroped until the vapor temperature is between about 90° C. and about 100° C.

After heating, the solution is allowed to cool, preferably between ambient temperature and about 0° C., optionally with chilling. Preferably, the solution is cooled to ambient temperature. Cooling allows the precipitation of the selected polymorph of zolpidem hemitartrate. The cooled solution can be allowed to digest at ambient temperature or lower while crystals continue to form to increase yield.

After cooling and precipitation of the selected polymorph, the solid may be isolated by filtration, centrifugation, distilling to dryness, decanting, or spray drying. The solids are preferentially dried in an oven to remove substantially all of the residual solvents. Drying can occur at a temperature between about 25° C. and about 55° C., more preferentially between about 35° C. and about 45° C. Drying can occur under ambient pressure, but more preferably, drying occurs in a vacuum oven at a pressure between about 5 millibar (mb) and about 60 mb, more preferably between about 25 mb and about 30 mb.

According to the process outlined above, selected polymorphs of zolpidem hemitartrate can be prepared directly from zolpidem free base. Advantageously, the preparation of a polymorph according to the present invention avoids the isolation of the desired polymorph from a mixture of polymorphs which are prepared according to methods known in the art.

The following examples further illustrate the process of the present invention.

### Example 1

### Selective Preparation of Polymorph E

Zolpidem free base (5 g) was dissolved in methanol (36 mL), yielding a first solution. L-(+)-tartaric acid (1.2 g) was dissolved in methanol (12 mL), yielding a second solution. The first and second solutions were combined in a flask to yield a feed solution, which was stirred at 65° C. for 30 minutes. Water (50 mL) was added to the feed solution. This feed solution was distilled until 50 mL of distillate evaporated from the feed solution. This distillation left a feed solution having less than 30% methanol remaining. The remaining feed solution was cooled to ambient temperature with stirring until solid product precipitated from the feed solution. The solution was then rotary evaporated to dryness at a pressure of 100 mb and a temperature of 40° C., leaving a dry powder. The dry powder was removed from the flask by adding 100 mL water. The flask was scraped to remove as much powder as possible. The zolpidem hemitartrate suspension was filtered in a vacuum funnel, and the powder was collected in the filter. The powder was dried in a vacuum oven for 3 days at 40° C. The dried solid was ground in a mortar and pestle. The powdered solid weighed 3.48 g (56% yield).

The powder was prepared for pXRD analysis. The pXRD pattern indicated that the product comprised predominantly polymorph E. The filtrate was recycled for the next batch to minimize the use of water and loss of zolpidem hemitartrate.

### Example 2

### Selective Preparation of Polymorph E

Zolpidem free base (5 g) was dissolved in methanol (36 mL), yielding a first solution. L-(+)-tartaric acid (1.2 g) was dissolved in methanol (12 mL), yielding a second solution. The first and second solutions were combined to yield a feed solution, which was stirred at 65° C. for 30 minutes. This feed solution was distilled at a temperature of 75° C. until 15 mL of distillate evaporated from the feed solution. Water (25 mL) was then added to the feed solution, which was further distilled until the vapor temperature reached 94° C. At this point, about 30 mL additionally distilled such that the final volume of distillate collected was 45 mL. The remaining feed solution was cooled to ambient temperature with stirring until a solid product precipitated from the feed solution. The zolpidem hemitartrate suspension was filtered in a vacuum funnel, and the powder was collected in the filter. The filtrate was saved for later use. The powder was dried in a vacuum oven at 50° C. overnight. The dried solid was ground in a mortar and pestle.

The white powder was prepared for pXRD analysis. The pXRD pattern indicated that the product comprised predominantly polymorph E. The filtrate was recycled for a second preparation, i.e., instead of adding 25 mL of water to the feed solution as in the above preparation, the filtrate from the first preparation was added to a methanol solution comprising zolpidem and L-tartaric acid.

The first batch of white powder weighed 5.00 g, representing an 81% yield of polymorph E. The second batch of white powder (recovered from a second 5.0 g batch of Zolpidem free base which was distilled using the filtrate from the first process) weighed 6.63 g. Accordingly, the total yield from both first and second preparations was about 94%.

### Example 3

### Selective Preparation of Polymorph H

Zolpidem base (1 g) and L-(+)-tartaric acid (0.24 g) were dissolved in ethanol (10 mL). The solution was stirred at 60° C. until all solids dissolved. The solution was allowed to cool to ambient temperature, which caused a solid to precipitate and crystallize. The suspension was filtered, and the solid was dried under vacuum for 4 hours to obtain 1.0 g of powder (81% yield). The isolated powder was prepared for pXRD analysis, which indicated that the solid comprised polymorph H.

### Example 4

### Selective Preparation of Polymorph D

Zolpidem base (1 g) and L-(+)-tartaric acid (0.24 g) were dissolved in isopropanol (10 mL). The solution was heated to 70° C. and stirred until all solids dissolved. The solution was allowed to cool to ambient temperature, which caused a solid to precipitate and crystallize. The suspension was filtered, and the solid was dried under vacuum for 4 hours to obtain 0.71 g of white powder (57% yield). The isolated powder was prepared for pXRD analysis, which indicated that the solid comprised polymorph D.

### Example 5

### Selective Preparation of Polymorph D

Zolpidem base (1 g) and L-(+)-tartaric acid (0.24 g) were dissolved in an isopropanol:water solvent (90:10 by weight, 40 mL total volume). The solution was heated to boiling and stirred until all solids dissolved. The solution was allowed to cool to ambient temperature, which caused a solid to precipitate and crystallize. The suspension was filtered, and the solid was dried under vacuum for 4 hours to obtain 0.71 g of a white powder (57% yield). The isolated powder was prepared for pXRD analysis, which indicated that the solid comprised polymorph D.

### Example 6

### Selective Preparation of Polymorph D

Zolpidem base (1 g) and L-(+)-tartaric acid (0.24 g) were dissolved in methanol (10 mL) The solution was heated to 50° C. and stirred until all solids dissolved. Water (1 mL) was added. The solution was allowed to cool to ambient temperature, which caused a solid to precipitate and crystallize. The suspension was filtered, and the solid was dried under vacuum at 60° C. for 4 hours to obtain 0.32 g of a white powder (26% yield). The isolated powder was prepared for pXRD analysis, which indicated that the solid comprised polymorph D.

### Example 7

### Preparation of Polymorph C of Zolpidem Tartrate and Zolpidem Free Base

Hydrated Zolpidem hemitartrate (0.7 g, polymorph H) of Example 3 was dissolved in methanol (9 mL). The solution was heated to 50° C. and stirred until all solids dissolved. Water (2.5 mL) was added. The solution was allowed to cool to ambient temperature, which caused a solid to precipitate and crystallize. The suspension was filtered, and the solid was dried under vacuum at 60° C. for 4 hours to obtain a white powder. pXRD analysis indicated it was polymorph C of 1:1 (acid:base) salt of Zolpidem tartrate and Zolpidem free base. The purity was assayed by HPLC. The assay indicated purity between 89.9% to 92.4% of the novel zolpidem tartrate.

### Example 8

### Preparation of Polymorph C of Zolpidem Tartrate and Zolpidem Free Base

Zolpidem hemitartrate polymorph D (0.71 g) of Example 4 was dissolved in methanol (7 mL). The solution was heated to 50° C. and stirred until all solids dissolved. Water (3 mL) was added. The solution was allowed to cool to ambient temperature, which caused a solid to precipitate and crystallize. The suspension was filtered, and the solid was dried under vacuum at 60° C. overnight to obtain a white powder. pXRD analysis indicated that it was polymorph C of 1:1 (acid:base) salt of Zolpidem tartrate and Zolpidem free base.

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

1. A method for preparing a desired polymorph of a hemitartrate salt of a compound having the structure: 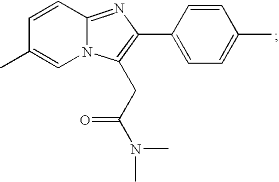
the method comprising:
dissolving a free base form of the compound in a liquid medium comprising an alcohol and a tartrate derivative to form a solution comprising the compound, the alcohol, and the tartrate derivative;
heating the solution to a temperature sufficient to dissolve the compound and the tartrate derivative;
and
cooling the solution to a temperature sufficient to precipitate the hemitartrate salt of the compound.

2. The method of claim 1 wherein the tartrate derivative is selected from the group consisting of L-(+)-tartaric acid, a monobasic salt of L-(+)-tartaric acid, a dibasic salt of L-(+)-tartaric acid, and combinations thereof.

3. The method of claim 2 wherein the alcohol is selected from the group consisting of methanol, ethanol, isopropanol, and n-propanol.

4. The method of claim 3 wherein the compound and the tartrate derivative are present in the solution at concentrations such that the molar ratio of the compound to the tartrate derivative is between about 2.5:1 and about 2:1.

5. The method of claim 4 wherein the compound is present in the solution at a concentration between about 0.30 M and about 0.35 M.

6. The method of claim 5 wherein the liquid medium further comprises water.

7. The method of claim 6 wherein the solution is heated to a temperature between about 25° C. and about 85° C.

8. The method of claim 7 wherein the solution is cooled to a temperature between about 0° C. and about 25° C.

9. The method of claim 8 further comprising the step of distilling a volume of the liquid medium.

10. The method of claim 9 wherein distilling occurs until the distillate vapor reaches a temperature between about 90° C. and about 100° C.

11. The method of claim 8 further comprising the step of rotary evaporation of a volume of the liquid medium.

12. The method of claim 8 further comprising the step of azeotroping a volume of the liquid medium.

13. The method of claim 12 further comprising the step of agitating the solution.

14. The method of claim 13 wherein the alcohol is methanol.

15. The method of claim 13 wherein the alcohol is ethanol.

16. The method of claim 13 wherein the alcohol is isopropanol.

17. The method of claim 13 wherein the alcohol is n-propanol.

18. The method of claim 17 further comprising isolating the precipitated hemitartrate salt of the compound by filtering, centrifugation, decanting, distilling to dryness, and spray drying.
